(19) United States
(12) Patent Application Publication
(10) Pub. No.: US 2015/0132244 A1
(43) Pub. Date: May 14, 2015

(54) AGENT AND METHOD FOR THE TEMPORARY DEFORMATION OF KERATIN FIBRES

(71) Applicant: Henkel AG & Co. KGaA, Duesseldorf (DE)

(72) Inventors: Thorsten Knappe, Schenefeld (DE); Anna Henschel, Winsen (Luhe) (DE)

(21) Appl. No.: 14/592,003

(22) Filed: Jan. 8, 2015

Related U.S. Application Data
(63) Continuation of application No. PCT/EP2013/063592, filed on Jun. 28, 2013.

(30) Foreign Application Priority Data
Jul. 9, 2012 (DE) 10 2012 211 912.4

(51) Int. Cl. A61K 8/891; A61K 8/42; A61K 8/34; A61Q 5/06

(52) U.S. Cl. CPC A61K 8/891; A61Q 5/06; A61K 8/42; A61K 8/345; A61K 2800/59; A61K 2800/48
USPC 424/70.12

(57) ABSTRACT

Powdered cosmetic compositions containing a) 5 wt % to 20 wt % hydrophobized metal oxide powder, b) 40 wt % to 94 wt % organic polyol, c) 0.1 wt % to 15 wt % emulsifier agent, d) 0 to 40 wt % water are particularly suitable for reshaping keratinic fibers, and impart shine, texture, and smoothness.

### FIELD OF THE INVENTION

The present invention generally relates to the technical field of the temporary reshaping of keratin-containing fibers, in particular human hair.

### BACKGROUND OF THE INVENTION

Styling agents for the reshaping of keratin-containing fibers have been known for some time and are utilized in various embodiments to construct, refresh, and retain hairstyles that, for many types of hair, can be obtained only with the use of setting active agents. An important role is played here by hair treatment agents that serve for both permanent and temporary shaping of the hair. Temporary shaping results that are to exhibit good hold without negatively affecting the healthy appearance of the hair, for example its shine, can be achieved e.g. using hair sprays, hair waxes, hair gels, blow-dry waves, etc.

Corresponding agents for temporary shaping usually contain synthetic polymers as a shaping component. Preparations that contain a polymer can be applied onto the hair by means of propellant gases or with a pump mechanism. Hair gels and hair waxes, on the other hand, are as a rule applied onto the hair not directly but instead by means of a comb or the hands.

Known forms of temporary styling agents can often not be metered with satisfactory accuracy. Hair gels, hair creams, and hair waxes, for example, are difficult to distribute once they have been applied onto the hair.

Hair sprays can be distributed more uniformly onto the hair. But because the user has no ability to visually perceive the total quantity of styling agent applied, the risk exists that more styling agent than would actually be necessary is applied onto the hair.

Powdered cosmetics are known and have already been used for some time, for example, in the skin treatment sector. Typical examples are make-up powder or eye shadow. The use of a powdered carrier material is necessary in order to achieve the powdered consistency. A metal oxide, for example silicon dioxide, can be used as a suitable carrier material. Hydrophobized metal oxide or silicon dioxide is of particular interest. This can be obtained, for example, from pyrogenic silicon dioxide, which is commercially obtainable in various specifications. Untreated pyrogenic silicon dioxide carries silanol groups and siloxane groups on the surface. As a result, it has a high affinity for water, i.e. it is hydrophilic. By reaction with suitable organic silicon compounds, alkylsilyl groups chemically bond to the surface of the pyrogenic silicon dioxide. This results in modified silicon dioxide powders that can no longer be wetted by water, i.e. that have hydrophobic properties. These hydrophobized silicon dioxides are suitable for manufacturing so-called “dry water,” in which the water droplets are prevented from flowing back together. The resulting powdered solids can have a water content of up to 95%. Under mechanical stress, for example when rubbed onto the skin, the enclosed water is released again.

Cosmetic or pharmaceutical liquefiable powder compositions are described, for example, in European patent EP 1 235 554 B1.

International application WO 03/037287 A1 discloses the use of a granulate based on pyrogenic silicon dioxide in cosmetic compositions. The special granulates can be silanized, i.e. hydrophobized, and are suitable for manufacturing cosmetic compositions of any consistency, for example liquids, foams, sprays, or powders.

International application WO 2007/051511 A1 describes the use of a powdered composition, containing 50 to 95 wt % of an aqueous solvent, hydrophobized silicon dioxide powder, and a film-forming and/or setting polymer present at least in the aqueous solvent, for the temporary reshaping of keratinic fibers.

The subject of German patent application DE 10 2008 057261 A1 is powdered compositions that are used for the temporary reshaping of hair for a very strong retaining hold on the hairstyle.

The powdered hair cosmetics of the existing art by now provide a hold that is acceptable for hair reshaping, and are notable for good metering characteristics. The result achieved with said agents, however, is capable of improvement in terms of the parameters of natural shine and the elasticity of the hold. In addition, most usual styling raw materials such as waxes, oils, or polymers are not readily suitable for manufacturing stable powdered compositions. Either they prevent successful formation of the core-shell particles, or the shelf stability of the core-shell particles that are formed is decreased.

The object of the present invention was therefore to make available shelf-stable powdered hair treatment agents for temporary shaping which can be metered accurately and simply, do not stick the hair together, and impart to the hair a fuller and natural feel and natural shine. There is to be no negative effect on the holding capability of the styling result.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A powdered cosmetic composition containing 5 to 20 wt % hydrophobized metal oxide powder, 40 to 94 wt % organic polyol, 0.1 to 15 wt % emulsifier agent, and 0 to 40 wt % water.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has been found that powdered cosmetic compositions based on a polyol/emulsifier agent mixture achieve the aforesaid object. A first subject of the present application is powdered cosmetic compositions containing

a) 5 to 20 wt % hydrophobized metal oxide powder,
b) 40 to 94 wt % organic polyol,
c) 0.1 to 15 wt % emulsifier agent,
d) 0 to 40 wt % water.

The powdered compositions according to the present invention are present preferably in the form of core-shell particles whose shell contains particles of at least one hydrophobized metal oxide powder as well as the emulsifier agent, and whose liquid core contains organic polyol as well as optionally water.

“Particles” are, for purposes of the invention, particles (cf. DIN 66160: 1992-09) of solids, which are present as grains.

Compositions whose particles are freely pourable under their own weight (cf. DIN EN ISO 6186: 1998-08) are “powdered” for purposes of the invention.

The powdered compositions according to the present invention are notable for the fact that as a result of a mechanical stress on the core-shell particles, in particular as a result of friction and/or pressure, the liquid core becomes released from the core-shell particle and a liquid thereby forms from the powdered composition. This is thus a powdered powder-to-liquid composition. The powdered compositions according to the present invention can be metered very easily. They can furthermore be distributed very uniformly in hair, since the liquid core is released only under mechanical stress at the site of action, and controlled wetting of the hair fibers is enabled. The powder can thus firstly be carefully distributed in the hair and only then more strongly mechanically stressed, for example by deliberately massaging the powder into the hair. The styling effect is thus produced only directly on the desired hair area.

The powdered compositions that are used contain hydrophobized metal oxide. Preferred compositions are characterized in that they contain the hydrophobized metal oxide powder in quantities, based on their total weight, from 5.5 to 18, preferably from 6 to 15 wt %. The optimum quantity depends chiefly on the hydrophobicity of the silicone dioxide powder that is used. The more hydrophobic the silicon dioxide powder, the less of it is required in order to obtain a stable powdered product.

The nature of the hydrophobized metal oxide is in principle not limited, provided there is assurance that a powdered product is produced upon intensive mixing with the liquid aqueous phase. Those metal oxides which have been modified, at least on the surface of the particles, in such a way that the modified particles are wetted less by water than the unmodified particle, are to be understood as “hydrophobized” for purposes of the invention. Silanized hydrophobized metal oxides are particularly preferred. At least one representative of the group that is constituted from silanes, halosilanes, alkoxysilanes, and silazanes is preferably suitable according to the present invention as a reagent for silanizing the metal oxide. Preferably suitable hydrophobized metal oxides of the hydrophobized metal oxide powder are selected according to the present invention from at least one representative of the group that is constituted from hydrophobized silicates, hydrophobized aluminum silicates, hydrophobized titanium dioxide, and hydrophobized silicon dioxide. Hydrophobized silicates have proven to be particularly suitable for manufacturing the cosmetic compositions according to the present invention; pyrogenic silicic acid post-treated with polydimethylsiloxane exhibits particular advantages. Preferred compositions according to the present invention are therefore characterized in that they contain as a hydrophobized metal oxide powder a hydrophobized silicate, preferably a pyrogenic silicic acid post-treated with polydimethylsiloxane. Corresponding metal oxides having the INCI name Silica Dimethicone Silylate are marketed, for example, by the Evonik Company under the commercial name Aerosil® R202.

The particle diameter of the primary particles of preferred hydrophobized metal oxides is preferably less than 5 μm, particularly preferably less than 1 μm, and in particular between 1 and 50 nm.

Also preferred are those hydrophobized silicon dioxides which have a specific surface area according to BET of between 10 and 400 m²/g, preferably between 40 and 300 m²/g, and in particular 80 to 150 m²/g.

The powdered compositions according to the present invention contain an organic polyol as a second essential constituent. Preferred compositions contain the organic polyol in quantities, based on their total weight, from 45 to 92 wt %, preferably from 60 to 90 wt %, and in particular from 70 to 88 wt %. The polyol can be used as an individual substance or in the form of polyol mixtures. Preferred agents according to the present invention are notable for the fact that they contain fewer than four, preferably one to three, but in particular only one polyol. Polyols from the group of glycerol, 1,2-ethanediol, polyethylene glycols of MW>400, propanediol, butanediol, in particular 1,3-butanediol, hexanediol, in particular 1,6-hexanediol, sorbitol, threitol, erythritol, arabitol, altritol, ribitol, xylitol, galactitol, mannitol, iditol, and panthenol are particularly suitable for the manufacture of cosmetic compositions. Glycerol, sorbitol, and panthenol are particularly suitable organic polyols. These polyols can be converted into a powdered form using comparatively small quantities of hydrophobized metal oxide powder, as a rule with less than 10 wt % metal oxide powder (based on the total weight of the powdered cosmetic composition).

Preferred compositions according to the present invention are therefore characterized in that they contain as an organic polyol at least one compound from the group of glycerol, sorbitol, and panthenol.

A further essential constituent of the compositions according to the present invention is an emulsifier agent. Preferred compositions contain the emulsifier agent in quantities, based on their total weight, from 1.0 to 12 wt %, preferably 2.0 to 11 wt %, and in particular from 5.0 to 10 wt %.

The cosmetic agents furthermore preferably contain at least one representative from the group of anionic, amphoteric, zwitterionic, nonionic, cationic emulsifier agents or mixtures thereof. It is preferred to use emulsifiers that are solid at room temperature (20° C.), in particular anionic emulsifier agents that are solid at room temperature (20° C.).

Anionic emulsifier agents are characterized by a hydrophilic anionic group, for example a carboxylate, sulfate, sulfonate, or phosphate group, and a lipophilic alkyl group. Glycol ether or polyglycol ether groups, ester, ether, and amide groups, and hydroxyl groups can additionally be contained in the molecule. Examples of suitable anionic surfactants and emulsifiers are, in each case in the form of the sodium, potassium, and ammonium and mono-, di, and trialkanolammonium salts having 2 to 4 carbon atoms in the alkanol group:
linear and branched fatty acids having 8 to 30 carbon atoms (soaps);
ether carboxylic acids of the formula R—O—(CH₂—CH₂O)ₓ—CH₂—COOH, in which R is a linear alkyl group having 8 to 30 carbon atoms and x=0 or is 1 to 16;
acyl sarcosides having 8 to 24 carbon atoms in the acyl group;
acyl taurides having 8 to 24 carbon atoms in the acyl group;
acyl isethionates having 8 to 24 carbon atoms in the acyl group;
linear alkanesulfonates having 8 to 24 carbon atoms;
linear alpha-olefinsulfonates having 8 to 24 carbon atoms;
alpha-sulfo fatty acid methyl esters of fatty acids having 8 to 30 carbon atoms;
acyl glutamates, in particular sodium-N-cocoyl- and sodium-N-stearoyl-L-glutamate;
esters of sulfosuccinic acid or of sulfosuccinates, of the general formula (T3I)

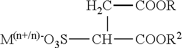

in which M^{(n+/n)} for n=1 denotes a hydrogen atom, an alkali-metal cation, an ammonium group, or the cation of an organoammonium base, and for n=2 represents an alkaline-earth-metal cation, and R¹ and R² mutually independently denote a hydrogen atom, an alkali-metal or alkaline-earth-metal cation, an ammonium group, the cation of an organoammonium base, or a residue Z that derives from a polyhydroxylated organic compound that is selected from the group of etherified (C₆ to C₁₈) alkylpolysaccharides having 1 to 6 monomeric saccharide units and/or etherified aliphatic (C₆ to C₁₆) hydroxyalkyl polyols having 2 to 16 hydroxyl residues, with the provision that at least one of the groups R¹ or R² is a residue Z;
sulfosuccinic acid mono- and dialkyl esters having 8 to 24 carbon atoms in the alkyl group, and sulfosuccinic acid monoalkylpolyoxyethyl esters having 8 to 24 carbon atoms in the alkyl group and 1 to 6 oxyethyl groups;
alkyl sulfates and alkyl polyglycol ether sulfates of the formula R—(O—CH₂—CH₂)ₓ—OSO₃H, in which R is a preferably linear alkyl group having 8 to 30 carbon atoms, and x=0 or is 1 to 12;
mixed surface-active hydroxysulfonates according to DE-A-37 25 030;
esters of tartaric acid and citric acid with alcohols that represent addition products of approximately 2 to 15 molecules of ethylene oxide and/or propylene oxide with C₈₋₂₂ fatty alcohols;
alkyl and/or alkenyl ether phosphates;
sulfated fatty acid alkylene glycol esters;
monoglyceride sulfates and monoglyceride ether sulfates.

Particularly preferred anionic emulsifier agents are cetearyl sulfosuccinates and stearoyl glutamates.

In summary, preferred cosmetic compositions according to the present invention contain as an emulsifier agent an emulsifier agent that is solid at room temperature (20° C.), particularly preferably an anionic emulsifier agent that is solid at room temperature (20° C.), in particular at least one emulsifier agent from the group of cetearyl succinates and stearoyl glutamates.

Lastly, the cosmetic compositions according to the present invention can also contain water. The production of preparations low in water has, however, proven to be advantageous for the cosmetic properties of the preparations according to the present invention. Cosmetic compositions according to the present invention are therefore characterized in that they contain, based on their total weight, less than 25 wt %, preferably less than 10 wt %, by preference less than 5.0 wt %, and in particular less than 1.0 wt % water.

The composition of some preferred cosmetic agents may be gathered from the tables that follow (unless otherwise indicated, values are in wt % based on the total weight of the cosmetic agent).

| | Formula 1 | Formula 2 | Formula 3 | Formula 4 | Formula 5 |
|---|---|---|---|---|---|
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Polyol | 40 to 94 | 45 to 92 | 60 to 90 | 70 to 88 | 70 to 88 |
| Emulsifier | 0.1 to 15 | 1.0 to 12 | 2.0 to 11 | 5.0 to 10 | 5.0 to 10 |
| agent | | | | | |
| Water | 0 to 40 | 0 to 25 | 0 to 10 | <1.0 | — |

| | | | | | Formula |
|---|---|---|---|---|---|
| | Formula 6 | Formula 7 | Formula 8 | Formula 9 | 10 |
| Silica | 5 to 20 | 5.5 to 18 | 6.0 to 15 | 6.0 to 10 | 6.0 to 10 |
| Dimethicone | | | | | |
| Silylate | | | | | |
| Polyol | 40 to 94 | 45 to 92 | 60 to 90 | 70 to 88 | 70 to 88 |
| Emulsifier | 0.1 to 15 | 1.0 to 12 | 2.0 to 11 | 5.0 to 10 | 5.0 to 10 |
| agent | | | | | |
| Water | 0 to 40 | 0 to 25 | 0 to 10 | <1.0 | — |

| | Formula | Formula | Formula | Formula | Formula |
|---|---|---|---|---|---|
| | 11 | 12 | 13 | 14 | 15 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Glycerol | 40 to 94 | 45 to 92 | 60 to 90 | 70 to 88 | 70 to 88 |
| Emulsifier | 0.1 to 15 | 1.0 to 12 | 2.0 to 11 | 5.0 to 10 | 5.0 to 10 |
| agent | | | | | |
| Water | 0 to 40 | 0 to 25 | 0 to 10 | <1.0 | — |

| | Formula | Formula | Formula | Formula | Formula |
|---|---|---|---|---|---|
| | 16 | 17 | 18 | 19 | 20 |
| Silica | 5 to 20 | 5.5 to 18 | 6.0 to 15 | 6.0 to 10 | 6.0 to 10 |
| Dimethicone | | | | | |
| Silylate | | | | | |
| Glycerol | 40 to 94 | 45 to 92 | 60 to 90 | 70 to 88 | 70 to 88 |
| Emulsifier | 0.1 to 15 | 1.0 to 12 | 2.0 to 11 | 5.0 to 10 | 5.0 to 10 |
| agent | | | | | |
| Water | 0 to 40 | 0 to 25 | 0 to 10 | <1.0 | — |

| | Formula | Formula | Formula | Formula | Formula |
|---|---|---|---|---|---|
| | 21 | 22 | 23 | 24 | 25 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Sorbitol | 40 to 94 | 45 to 92 | 60 to 90 | 70 to 88 | 70 to 88 |
| Emulsifier | 0.1 to 15 | 1.0 to 12 | 2.0 to 11 | 5.0 to 10 | 5.0 to 10 |
| agent | | | | | |
| Water | 10 to 40 | 10 to 25 | 10 to 25 | 10 to 25 | 15 to 25 |

| | Formula | Formula | Formula | Formula | Formula |
|---|---|---|---|---|---|
| | 26 | 27 | 28 | 29 | 30 |
| Silica | 5 to 20 | 5.5 to 18 | 6.0 to 15 | 6.0 to 10 | 6.0 to 10 |
| Dimethicone | | | | | |
| Silylate | | | | | |
| Sorbitol | 40 to 94 | 45 to 92 | 60 to 90 | 70 to 88 | 70 to 88 |
| Emulsifier | 0.1 to 15 | 1.0 to 12 | 2.0 to 11 | 5.0 to 10 | 5.0 to 10 |
| agent | | | | | |
| Water | 10 to 40 | 10 to 25 | 10 to 25 | 10 to 25 | 15 to 25 |

| | Formula | Formula | Formula | Formula | Formula |
|---|---|---|---|---|---|
| | 31 | 32 | 33 | 34 | 35 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Panthenol | 40 to 94 | 45 to 92 | 60 to 90 | 70 to 88 | 70 to 88 |
| Emulsifier | 0.1 to 15 | 1.0 to 12 | 2.0 to 11 | 5.0 to 10 | 5.0 to 10 |
| agent | | | | | |
| Water | 0 to 40 | 0 to 25 | 0 to 10 | <1.0 | — |

| | Formula | Formula | Formula | Formula | Formula |
|---|---|---|---|---|---|
| | 36 | 37 | 38 | 39 | 40 |
| Silica | 5 to 20 | 5.5 to 18 | 6.0 to 15 | 6.0 to 10 | 6.0 to 10 |
| Dimethicone | | | | | |
| Silylate | | | | | |
| Panthenol | 40 to 94 | 45 to 92 | 60 to 90 | 70 to 88 | 70 to 88 |
| Emulsifier | 0.1 to 15 | 1.0 to 12 | 2.0 to 11 | 5.0 to 10 | 5.0 to 10 |
| agent | | | | | |
| Water | 0 to 40 | 0 to 25 | 0 to 10 | <1.0 | — |

| | Formula | Formula | Formula | Formula | Formula |
|---|---|---|---|---|---|
| | 41 | 42 | 43 | 44 | 45 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Polyol | 40 to 94 | 45 to 92 | 60 to 90 | 70 to 88 | 70 to 88 |
| Anionic | 0.1 to 15 | 1.0 to 12 | 2.0 to 11 | 5.0 to 10 | 5.0 to 10 |
| emulsifier | | | | | |
| agent | | | | | |
| Water | 0 to 40 | 0 to 25 | 0 to 10 | <1.0 | — |

| | Formula | Formula | Formula | Formula | Formula |
|---|---|---|---|---|---|
| | 46 | 47 | 48 | 49 | 50 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Polyol | 40 to 94 | 45 to 92 | 60 to 90 | 70 to 88 | 70 to 88 |
| Cetearyl | 0.1 to 15 | 1.0 to 12 | 2.0 to 11 | 5.0 to 10 | 5.0 to 10 |
| sulfo- | | | | | |
| succinate | | | | | |
| Water | 0 to 40 | 0 to 25 | 0 to 10 | <1.0 | — |

| | Formula | Formula | Formula | Formula | Formula |
|---|---|---|---|---|---|
| | 51 | 52 | 53 | 54 | 55 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Polyol | 40 to 94 | 45 to 92 | 60 to 90 | 70 to 88 | 70 to 88 |
| Stearoyl | 0.1 to 15 | 1.0 to 12 | 2.0 to 11 | 5.0 to 10 | 5.0 to 10 |
| glutamate | | | | | |
| Water | 0 to 40 | 0 to 25 | 0 to 10 | <1.0 | — |

| | Formula | Formula | Formula | Formula | Formula |
|---|---|---|---|---|---|
| | 56 | 57 | 58 | 59 | 60 |
| Silica | 5 to 20 | 5.5 to 18 | 6.0 to 15 | 6.0 to 10 | 6.0 to 10 |
| Dimethicone | | | | | |
| Silylate | | | | | |
| Polyol | 40 to 94 | 45 to 92 | 60 to 90 | 70 to 88 | 70 to 88 |
| Anionic | 0.1 to 15 | 1.0 to 12 | 2.0 to 11 | 5.0 to 10 | 5.0 to 10 |
| emulsifier | | | | | |
| agent | | | | | |
| Water | 0 to 40 | 0 to 25 | 0 to 10 | <1.0 | — |

| | Formula | Formula | Formula | Formula | Formula |
|---|---|---|---|---|---|
| | 61 | 62 | 63 | 64 | 65 |
| Silica | 5 to 20 | 5.5 to 18 | 6.0 to 15 | 6.0 to 10 | 6.0 to 10 |
| Dimethicone | | | | | |
| Silylate | | | | | |
| Polyol | 40 to 94 | 45 to 92 | 60 to 90 | 70 to 88 | 70 to 88 |
| Cetearyl | 0.1 to 15 | 1.0 to 12 | 2.0 to 11 | 5.0 to 10 | 5.0 to 10 |
| sulfo- | | | | | |
| succinate | | | | | |
| Water | 0 to 40 | 0 to 25 | 0 to 10 | <1.0 | — |

| | Formula | Formula | Formula | Formula | Formula |
|---|---|---|---|---|---|
| | 66 | 67 | 68 | 69 | 70 |
| Silica | 5 to 20 | 5.5 to 18 | 6.0 to 15 | 6.0 to 10 | 6.0 to 10 |
| Dimethicone | | | | | |
| Silylate | | | | | |
| Polyol | 40 to 94 | 45 to 92 | 60 to 90 | 70 to 88 | 70 to 88 |
| Stearoyl | 0.1 to 15 | 1.0 to 12 | 2.0 to 11 | 5.0 to 10 | 5.0 to 10 |
| glutamate | | | | | |
| Water | 0 to 40 | 0 to 25 | 0 to 10 | <1.0 | — |

| | Formula | Formula | Formula | Formula | Formula |
|---|---|---|---|---|---|
| | 71 | 72 | 73 | 74 | 75 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Glycerol | 40 to 94 | 45 to 92 | 60 to 90 | 70 to 88 | 70 to 88 |
| Anionic | 0.1 to 15 | 1.0 to 12 | 2.0 to 11 | 5.0 to 10 | 5.0 to 10 |
| emulsifier | | | | | |
| agent | | | | | |
| Water | 0 to 40 | 0 to 25 | 0 to 10 | <1.0 | — |

| | Formula | Formula | Formula | Formula | Formula |
|---|---|---|---|---|---|
| | 76 | 77 | 78 | 79 | 80 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Sorbitol | 40 to 94 | 45 to 92 | 60 to 90 | 70 to 88 | 70 to 88 |
| Anionic | 0.1 to 15 | 1.0 to 12 | 2.0 to 11 | 5.0 to 10 | 5.0 to 10 |
| emulsifier | | | | | |
| agent | | | | | |
| Water | 10 to 40 | 10 to 25 | 10 to 25 | 10 to 25 | 15 to 25 |

| | Formula | Formula | Formula | Formula | Formula |
|---|---|---|---|---|---|
| | 81 | 82 | 83 | 84 | 85 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Panthenol | 40 to 94 | 45 to 92 | 60 to 90 | 70 to 88 | 70 to 88 |
| Anionic | 0.1 to 15 | 1.0 to 12 | 2.0 to 11 | 5.0 to 10 | 5.0 to 10 |
| emulsifier | | | | | |
| agent | | | | | |
| Water | 0 to 40 | 0 to 25 | 0 to 10 | <1.0 | — |

| | Formula | Formula | Formula | Formula | Formula |
|---|---|---|---|---|---|
| | 86 | 87 | 88 | 89 | 90 |
| Silica | 5 to 20 | 5.5 to 18 | 6.0 to 15 | 6.0 to 10 | 6.0 to 10 |
| Dimethicone | | | | | |
| Silylate | | | | | |
| Glycerol | 40 to 94 | 45 to 92 | 60 to 90 | 70 to 88 | 70 to 88 |
| Anionic | 0.1 to 15 | 1.0 to 12 | 2.0 to 11 | 5.0 to 10 | 5.0 to 10 |
| emulsifier | | | | | |
| agent | | | | | |
| Water | 0 to 40 | 0 to 25 | 0 to 10 | <1.0 | — |

| | Formula | Formula | Formula | Formula | Formula |
|---|---|---|---|---|---|
| | 91 | 92 | 93 | 94 | 95 |
| Silica | 5 to 20 | 5.5 to 18 | 6.0 to 15 | 6.0 to 10 | 6.0 to 10 |
| Dimethicone | | | | | |
| Silylate | | | | | |
| Sorbitol | 40 to 94 | 45 to 92 | 60 to 90 | 70 to 88 | 70 to 88 |
| Anionic | 0.1 to 15 | 1.0 to 12 | 2.0 to 11 | 5.0 to 10 | 5.0 to 10 |
| emulsifier | | | | | |
| agent | | | | | |
| Water | 10 to 40 | 10 to 25 | 10 to 25 | 10 to 25 | 15 to 25 |

| | Formula | Formula | Formula | Formula | Formula |
|---|---|---|---|---|---|
| | 96 | 97 | 98 | 99 | 100 |
| Silica | 5 to 20 | 5.5 to 18 | 6.0 to 15 | 6.0 to 10 | 6.0 to 10 |
| Dimethicone | | | | | |
| Silylate | | | | | |
| Panthenol | 40 to 94 | 45 to 92 | 60 to 90 | 70 to 88 | 70 to 88 |
| Anionic | 0.1 to 15 | 1.0 to 12 | 2.0 to 11 | 5.0 to 10 | 5.0 to 10 |
| emulsifier | | | | | |
| agent | | | | | |
| Water | 0 to 40 | 0 to 25 | 0 to 10 | <1.0 | — |

| | Formula | Formula | Formula | Formula | Formula |
|---|---|---|---|---|---|
| | 101 | 102 | 103 | 104 | 105 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Glycerol | 40 to 94 | 45 to 92 | 60 to 90 | 70 to 88 | 70 to 88 |
| Cetearyl | 0.1 to 15 | 1.0 to 12 | 2.0 to 11 | 5.0 to 10 | 5.0 to 10 |
| sulfo- | | | | | |
| succinate | | | | | |
| Water | 0 to 40 | 0 to 25 | 0 to 10 | <1.0 | — |

| | Formula | Formula | Formula | Formula | Formula |
|---|---|---|---|---|---|
| | 106 | 107 | 108 | 109 | 110 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Sorbitol | 40 to 94 | 45 to 92 | 60 to 90 | 70 to 88 | 70 to 88 |
| Cetearyl | 0.1 to 15 | 1.0 to 12 | 2.0 to 11 | 5.0 to 10 | 5.0 to 10 |
| sulfo- | | | | | |
| succinate | | | | | |
| Water | 10 to 40 | 10 to 25 | 10 to 25 | 10 to 25 | 15 to 25 |

| | Formula | Formula | Formula | Formula | Formula |
|---|---|---|---|---|---|
| | 111 | 112 | 113 | 114 | 115 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Panthenol | 40 to 94 | 45 to 92 | 60 to 90 | 70 to 88 | 70 to 88 |
| Cetearyl | 0.1 to 15 | 1.0 to 12 | 2.0 to 11 | 5.0 to 10 | 5.0 to 10 |
| sulfo- | | | | | |
| succinate | | | | | |
| Water | 0 to 40 | 0 to 25 | 0 to 10 | <1.0 | — |

| | Formula | Formula | Formula | Formula | Formula |
|---|---|---|---|---|---|
| | 116 | 117 | 118 | 119 | 120 |
| Silica | 5 to 20 | 5.5 to 18 | 6.0 to 15 | 6.0 to 10 | 6.0 to 10 |
| Dimethicone | | | | | |
| Silylate | | | | | |
| Glycerol | 40 to 94 | 45 to 92 | 60 to 90 | 70 to 88 | 70 to 88 |
| Cetearyl | 0.1 to 15 | 1.0 to 12 | 2.0 to 11 | 5.0 to 10 | 5.0 to 10 |
| sulfo- | | | | | |
| succinate | | | | | |
| Water | 0 to 40 | 0 to 25 | 0 to 10 | <1.0 | — |

| | Formula | Formula | Formula | Formula | Formula |
|---|---|---|---|---|---|
| | 121 | 122 | 123 | 124 | 125 |
| Silica | 5 to 20 | 5.5 to 18 | 6.0 to 15 | 6.0 to 10 | 6.0 to 10 |
| Dimethicone | | | | | |
| Silylate | | | | | |
| Sorbitol | 40 to 94 | 45 to 92 | 60 to 90 | 70 to 88 | 70 to 88 |
| Cetearyl | 0.1 to 15 | 1.0 to 12 | 2.0 to 11 | 5.0 to 10 | 5.0 to 10 |
| sulfo- | | | | | |
| succinate | | | | | |
| Water | 10 to 40 | 10 to 25 | 10 to 25 | 10 to 25 | 15 to 25 |

| | Formula | Formula | Formula | Formula | Formula |
|---|---|---|---|---|---|
| | 126 | 127 | 128 | 129 | 130 |
| Silica | 5 to 20 | 5.5 to 18 | 6.0 to 15 | 6.0 to 10 | 6.0 to 10 |
| Dimethicone | | | | | |
| Silylate | | | | | |
| Panthenol | 40 to 94 | 45 to 92 | 60 to 90 | 70 to 88 | 70 to 88 |
| Cetearyl | 0.1 to 15 | 1.0 to 12 | 2.0 to 11 | 5.0 to 10 | 5.0 to 10 |
| sulfo- | | | | | |
| succinate | | | | | |
| Water | 0 to 40 | 0 to 25 | 0 to 10 | <1.0 | — |

| | Formula | Formula | Formula | Formula | Formula |
|---|---|---|---|---|---|
| | 131 | 132 | 133 | 134 | 135 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Glycerol | 40 to 94 | 45 to 92 | 60 to 90 | 70 to 88 | 70 to 88 |
| Stearoyl | 0.1 to 15 | 1.0 to 12 | 2.0 to 11 | 5.0 to 10 | 5.0 to 10 |
| glutamate | | | | | |
| Water | 0 to 40 | 0 to 25 | 0 to 10 | <1.0 | — |

| | Formula | Formula | Formula | Formula | Formula |
|---|---|---|---|---|---|
| | 136 | 137 | 138 | 139 | 140 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Sorbitol | 40 to 94 | 45 to 92 | 60 to 90 | 70 to 88 | 70 to 88 |
| Stearoyl | 0.1 to 15 | 1.0 to 12 | 2.0 to 11 | 5.0 to 10 | 5.0 to 10 |
| glutamate | | | | | |
| Water | 10 to 40 | 10 to 25 | 10 to 25 | 10 to 25 | 15 to 25 |

| | Formula | Formula | Formula | Formula | Formula |
|---|---|---|---|---|---|
| | 141 | 142 | 143 | 144 | 145 |
| Hydro- | 5 to 20 | 5.5 to 18 | 6.0 to 15 | 6.0 to 10 | 6.0 to 10 |
| phobized | | | | | |
| metal oxide | | | | | |
| powder | | | | | |
| Panthenol | 40 to 94 | 45 to 92 | 60 to 90 | 70 to 88 | 70 to 88 |
| Stearoyl | 0.1 to 15 | 1.0 to 12 | 2.0 to 11 | 5.0 to 10 | 5.0 to 10 |
| glutamate | | | | | |
| Water | 0 to 40 | 0 to 25 | 0 to 10 | <1.0 | — |

| | Formula | Formula | Formula | Formula | Formula |
|---|---|---|---|---|---|
| | 146 | 147 | 148 | 149 | 150 |
| Silica | 5 to 20 | 5.5 to 18 | 6.0 to 15 | 6.0 to 10 | 6.0 to 10 |
| Dimethicone | | | | | |
| Silylate | | | | | |
| Glycerol | 40 to 94 | 45 to 92 | 60 to 90 | 70 to 88 | 70 to 88 |
| Stearoyl | 0.1 to 15 | 1.0 to 12 | 2.0 to 11 | 5.0 to 10 | 5.0 to 10 |
| glutamate | | | | | |
| Water | 0 to 40 | 0 to 25 | 0 to 10 | <1.0 | — |

| | Formula | Formula | Formula | Formula | Formula |
|---|---|---|---|---|---|
| | 151 | 152 | 153 | 154 | 155 |
| Silica | 5 to 20 | 5.5 to 18 | 6.0 to 15 | 6.0 to 10 | 6.0 to 10 |
| Dimethicone | | | | | |
| Silylate | | | | | |
| Sorbitol | 40 to 94 | 45 to 92 | 60 to 90 | 70 to 88 | 70 to 88 |
| Stearoyl | 0.1 to 15 | 1.0 to 12 | 2.0 to 11 | 5.0 to 10 | 5.0 to 10 |
| glutamate | | | | | |
| Water | 10 to 40 | 10 to 25 | 10 to 25 | 10 to 25 | 15 to 25 |

| | Formula | Formula | Formula | Formula | Formula |
|---|---|---|---|---|---|
| | 156 | 157 | 158 | 159 | 160 |
| Silica | 5 to 20 | 5.5 to 18 | 6.0 to 15 | 6.0 to 10 | 6.0 to 10 |
| Dimethicone | | | | | |
| Silylate | | | | | |
| Panthenol | 40 to 94 | 45 to 92 | 60 to 90 | 70 to 88 | 70 to 88 |
| Stearoyl | 0.1 to 15 | 1.0 to 12 | 2.0 to 11 | 5.0 to 10 | 5.0 to 10 |
| glutamate | | | | | |
| Water | 0 to 40 | 0 to 25 | 0 to 10 | <1.0 | — |

The cosmetic compositions according to the present invention can contain further adjuvants, care-providing agents, and additives. The weight proportion of the further ingredients contained in the powdered compositions according to the present invention besides components a) to d), in particular of the further adjuvants, care-providing agents, and additives contained in said compositions, in terms of the total weight of the powdered compositions according to the present invention is preferably less than 10 wt %, by preference less than 5.0 wt %, particularly preferably less than 2.0 wt %, and in particular less than 1.0 wt %. The weight proportion of said adjuvants, care-providing agents, and additives in terms of the total weight of the cosmetic agents according to the present invention can be, for example, 0.001 to 2 wt %, in particular 0.01 to 0.5 wt %.

Preferred cosmetic agents according to the present invention contain at least one oily substance. Included among the natural and synthetic cosmetic oily substances are, for example, vegetable oils, liquid paraffin oils, isoparaffin oils, and synthetic hydrocarbons, silicone oils, as well as di-n-alkyl ethers having a total of between 12 and 36 carbon atoms, in particular 12 to 24 carbon atoms.

A first preferred group of oily substances is vegetable oils. Examples of such oils are amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, orange oil, palm oil, peach-kernel oil, rapeseed oil, rice oil, sea buckthorn pulp oil, sea buckthorn seed oil, sesame oil, shea butter, soy oil, sunflower oil, grapeseed oil, walnut oil, wheat germ oil, wild rose oil, and the liquid components of coconut oil. Also suitable, however, are other triglyceride oils such as the liquid components of beef tallow as well as synthetic triglyceride oils.

Preferred oily substances derive from the group of silicone oils. Included in particular in the group of silicone oils are dimethicones, which also encompass cyclomethicones, aminofunctional silicones, and dimethiconols. Dimethicones can be both linear and branched, as well as cyclic or cyclic and branched. Suitable silicone oils or silicone gums are, in particular, dialkyl- and alkylarylsiloxanes, for example dimethylpolysiloxane and methylphenylpolysiloxane, as well as alkoxylated, quaternized, or also anionic derivatives thereof. Cyclic and linear polydialkylsiloxanes, alkoxylated and/or aminated derivatives thereof, dihydroxypolydimethylsiloxanes, and polyphenylalkylsiloxanes are preferred.

Ester oils, i.e. esters of C₆ to C₃₀ fatty acids with C₂ to C₃₀ fatty alcohols, preferably monoesters of fatty acids with alcohols having 2 to 24 carbon atoms, for example isopropyl myristate (Rilanit® IPM), isononanoic acid C16-18 alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V), are further preferred oily substances.

Cosmetic compositions in which the weight proportion of oily substance in terms of the total weight of the composition is 0.01 to 5.0 wt %, preferably 0.02 to 4.0 wt %, and in particular 0.05 to 2.0 wt % have proven to be advantageous with regard to cosmetic effect.

The agent can contain a protein hydrolysate and/or a derivative thereof as a further care-providing substance. Protein hydrolysates are product mixtures obtained by the acid-, base-, or enzyme-catalyzed breakdown of proteins. The term “protein hydrolysates” is also understood according to the present invention to mean total hydrolysates as well as individual amino acids and derivatives thereof, as well as mixtures of different amino acids. The molecular weight of the protein hydrolysates usable according to the present invention is between 75 (the molecular weight of glycine) and 200,000; the molecular weight is preferably 75 to 50,000, and very particularly preferably 75 to 20,000 dalton.

The agent according to the present invention can furthermore contain at least one vitamin, one provitamin, one vitamin precursor, and/or a derivative thereof as a care-providing substance. Those vitamins, provitamins, and vitamin precursors which are usually assigned to groups A, B, C, E, F, and H are preferred according to the present invention.

The agents according to the present invention can furthermore contain at least one plant extract, but also mono- or oligosaccharides and/or lipids, as a care-providing substance.

The powdered compositions according to the present invention can contain at least one UV filter as a care-providing substance. The UV filters are contained in the powdered compositions according to the present invention usually in quantities from 0.01 to 5 wt % based on the powdered composition according to the present invention. Quantities from 0.1 to 2.5 wt % are preferred.

The powdered compositions according to the present invention can be formulated in any desired containers, provided there is assurance that mechanical stress on the powder upon removal of the composition does not result in liquefaction. Jars, bottles, or even Tetra Paks are suitable, for example; the container can be equipped, for example, with a pouring and metering apparatus.

A further subject of the present application is the use of a cosmetic composition according to the present invention for the temporary reshaping of keratin-containing fibers, in particular human hair. In the context of the use of the powdered composition for temporary reshaping of keratinic fibers, preferably firstly the desired quantity of powdered composition is removed from the container. The composition can be applied directly onto the keratinic fibers to be treated or else, for example, onto one's hand. In the first case the applied powder can be exposed, directly on the keratinic fibers, to a mechanical stress, for example by means of one's hands, with the result that the liquid aqueous phase is released directly on the fibers. If the powdered composition is first placed onto one's hand, it can then firstly be carefully distributed in the hair and only then in turn be subjected to greater mechanical stress, for example by controlled massaging of the powder into the hair. The liquid aqueous phase is thereby released on the hair.

It is of course also possible firstly to rub the powdered composition on one's hand and only then to apply the resulting liquid or pasty agent onto the keratinic fibers. This procedure is not preferred, however, since an essential advantage of the powdered consistency of the styling agent, namely good distribution capability, is thereby sacrificed. The powdered composition can of course also be applied using an aid, for example a paintbrush, a sponge, a cloth, a brush, or a comb.

A further subject of the present application is a method for the temporary reshaping of keratin-containing fibers, in particular human hair, in which method the keratinic fibers are acted upon by a cosmetic composition according to the present invention and their shape is temporarily retained, characterized in that before, during, or after application onto the keratinic fibers, a plastically deformable mass is formed from the cosmetic composition by the action of a force.

Also claimed, lastly, is a method for manufacturing a powdered cosmetic composition according to the present invention containing
a) 5 to 20 wt % hydrophobized metal oxide powder,
b) 40 to 94 wt % organic polyol,
c) 0.1 to 15 wt % emulsifier agent,
d) 0 to 40 wt % water,
comprising the steps of
i) mixing constituents b) and optionally d);
ii) introducing the hydrophobized metal oxide powder a) into the mixture resulting from step i) and mixing again, preferably for a time period from 1 to 120 seconds, preferably from 10 to 60 seconds, accompanied by formation of a powder;
iii) introducing the preferably powdered emulsifier agent into the powder produced in step ii) and mixing again, preferably for a time period from 1 to 120 seconds, preferably from 10 to 60 seconds.

### EXAMPLES

Powdered styling agents V1 to V7 were produced as described below (quantities in wt %):

| Raw materials | V1 | V2 | V3 | V4 | V5 | V6 | V7 |
|---|---|---|---|---|---|---|---|
| Aerosil ® R 202¹⁾ | 6.4 | 7.50 | 6.4 | 6.4 | 6.4 | 6.4 | 6.4 |
| D-Panthenol | — | — | — | 84.6 | 84.6 | — | — |
| Glycerol | — | 35.0 | 84.6 | | | 84.6 | |
| Neosorb 70/70²⁾ | 84.6 | 48.5 | | | | | 84.6 |
| Eumulgin Prisma³⁾ | — | — | — | — | 9.0 | 9.0 | 4.0 |
| Eumulgin SG⁴⁾ | 9.0 | 9.0 | 9.0 | 9.0 | — | — | 5.0 |

¹⁾Pyrogenic silicic acid post-treated with polydimethylsiloxane (INCI name: Silica Dimethicone Silylate)
²⁾Sorbitol, 70% in water
³⁾Disodium cetearyl sulfosuccinate
⁴⁾Sodium stearoyl glutamate

All the constituents except Aerosil® R 202 and the emulsifier agent were mixed in a vessel. The Aerosil® R 202 S hydrophobized silicon dioxide powder was added to this liquid. After a stirring time of 30 to 45 seconds in each case, a stable powder had respectively formed. The emulsifier agent was then distributed in the stable powder by stirring (approximately 15 seconds). The completed styling powder thereby obtained was decanted into polyethylene bottles.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A powdered cosmetic composition containing
a) 5 wt % to 20 wt % hydrophobized metal oxide powder,
b) 40 wt % to 94 wt % organic polyol,
c) 0.1 wt % to 15 wt % emulsifier agent,
d) 0 to 40 wt % water.

2. The composition according to claim 1, wherein the hydrophobized metal oxide powder comprises 5.5 wt % to 18 wt % of the total composition.

3. The composition according to claim 1, wherein the hydrophobized metal oxide powder comprises 6 wt % to 15 wt % of the total composition

4. The composition according to claim 1, wherein the hydrophobized metal oxide powder includes a hydrophobized silicate.

5. The composition according to claim 4, wherein the the hydrophobized silicate is a pyrogenic silicic acid post-treated with polydimethylsiloxane.

6. The composition according to claim 1, wherein the organic polyol comprises 45 wt % to 92 wt % of the total composition.

7. The composition according to claim 1, wherein the organic polyol comprises 60 wt % to 90 wt % of the total composition.

8. The composition according to claim 1, wherein the organic polyol comprises 70 wt % to 88 wt % of the total composition.

9. The composition according to claim 1, wherein the organic polyol includes at least one compound selected from the group consisting of glycerol, sorbitol, and panthenol.

10. The composition according to claim 1, wherein the emulsifier agent comprises 1.0 wt % to 12 wt % of the total composition.

11. The composition according to claim 1, wherein the emulsifier agent comprises 2.0 wt % to 11 wt % of the total composition.

12. The composition according to claim 1, wherein the emulsifier agent comprises 5.0 wt % to 10 wt % of the total composition.

13. The composition according to claim 1, wherein the emulsifier agent includes an emulsifier agent that is solid at 20° C.

14. The composition according to claim 1, wherein the emulsifier agent includes an anionic emulsifier agent that is solid at 20° C.

15. The composition according to claim 1, wherein the emulsifier agent is selected cetearyl sulfosuccinates or stearoyl glutamates.

16. The composition according to claim 1, wherein the water comprises less than 25 wt % of the total composition.

17. The composition according to claim 1, wherein the water comprises less than 10 wt % of the total composition.

18. The composition according to claim 1, wherein the water comprises less than 5.0 wt % of the total composition.

19. The composition according to claim 1, wherein the water comprises less than 1.0 wt % water of the total composition.

20. A method for the temporary reshaping of keratin-containing fibers, in particular human hair, in which method the keratinic fibers are acted upon by a cosmetic composition according to one of claim 1 and their shape is temporarily retained, wherein, during, or after application onto the keratinic fibers, a plastically deformable mass is formed from the cosmetic composition by the action of a force.
